(19) United States
(12) Patent Application Publication
(10) Pub. No.: US 2003/0165579 A1
(43) Pub. Date: Sep. 4, 2003

(54) Euphorbia antiquorum extract, a pharmaceutical composition containing the same and methods for treatment of cancers

(76) Inventors: Chih-Hui Lin, Taichung (TW); Wen-Ching Cheng, Taipei (TW)

Correspondence Address: LaRiviere, Grubman & Payne, LLP, P.O. Box 3140, Monterey, CA, 93942, US

(21) Appl. No.: 10/085,848

(22) Filed: Feb. 27, 2002

(51) Int. Cl. A61K 35/78

(52) U.S. Cl. 424/725

(57) ABSTRACT

An herbal extract of Euphorbia antiquorum is provided. The herbal extract of the invention demonstrates in vitro inhibition of growth of hepatoma cells, colorectal adenocarcinoma cells, monocyte-like lymphoma and leukemia cells. This invention also provides a pharmaceutical composition containing the same and a method for treatments for cancers.

### FIELD OF THE INVENTION

This invention relates to herbal extracts from Euphorbia antiquorum, and more particularly, to a single-herb based pharmaceutical composition and method having therapeutic effects for treating cancers. According to the invention, the extracts from Euphorbia antiquorum have been found to show inhibitory effect on several different cancer cell lines. The extracts are useful in effective treatment of cancers, particularly hepatoma, colon cancer, leukemia and lymphocytoma.

### BACKGROUND OF THE INVENTION

Cancer is the second leading cause of death in the United States (CA Cancer J. Clin. 43:7). The occurrence of cancer for any one individual is correlated with numerous risk factors. Some risk factors are believed to include age, genetics, diet and environmental exposure (e.g., to mutagenic chemicals, radiation, transforming viruses, etc.). According to an estimation by World Health Organization, about 10 million new cancer cases are now occurring annually around the world. The number is expected to reach 15 million by the year 2015, with two thirds of these new cases occurring in developing countries (World Health 48:22, 1995). For example, Hepatoma is much more prevalent in many of the developing countries than in the industrialized world. Although relatively uncommon in the Western world, an estimated 300 cases will occur annually in Missouri, USA. Statistics show that about 20,000 new patients are diagnosed every year in United States. It is not known exactly what causes hepatoma. However, certain risk factors have been identified. The risk factors are: a history of cirrhosis of the liver, certain types of viral hepatitis infection, chronic inflammatory conditions, exposure to the following substances aflatoxin, vinyl chloride, thorium dioxide, arsenic, long-term use of anabolic steroids and tabacco.

In general, surgical resection is the treatment of choice for patients with hepatoma and can be curative, provided the tumor is confined to the liver and is completely removed. Unfortunately, many patients are not candidates for resection due to large tumor size, multiple tumors or severe cirrhosis of the liver.

Since the majority of patients with hepatoma have an unresectable disease, numerous alternative treatments have been developed. Chemotherapy is just suitable for patients in whom the disease is not curable with surgery. However, many of the current anticancer drugs used in combination with chemotherapy are present with major toxicity. Depending on the drug or combination of drug used, systemic chemotherapy may result in one or more toxicities including hematologic, vascular, neural, gastrointestinal, renal, pulmonary, otologic and lethal. For example, Adriamycin (ADR), also called Doxorubicin, has been used for decades to control tumors. ADR shows about 15-23% remission effect and controls the disease for a space. However, ADR was shown to have adverse effects, i.e., an increased risk of heart problems.

5-FU has been another one of the most famous chemotherapy drugs for treating many cancers for decades; it binds to an enzyme inside of the cancer cells and thereby exerts its anticancer effect on the cells. However, 5-FU is irreversibly catabolized to dihydrofluorouracil (an inactive metabolite) by the enzyme dihydropyrimidine dehydrogenase (DPD). This catabolic pathway is a critical step in determining 5-FU availability for conversion to nucleotides and eventual incorporation into either RNA or DNA. The degree and severity of the side effects depend on the amount and schedule of the administration of 5-FU. The most common side effects include soreness of the mouth, difficulty swallowing, diarrhea, stomach ache, low white blood counts, low platelet counts, anemia, sensitive skin to sun exposure and excessive tear formation.

While new therapeutics are being developed and tested for efficacy against tumors, e.g., hepatoma, many of the currently available cancer treatments are relatively ineffective. A clinical trial was conducted to investigate the therapeutic effects on hepatoma patients by use of ADR, 5-FU and Mitomycin C. It has been reported that in 62 patients, after taking 5-FU by hepatic intra-arterial infusion, only 48% responded to the medicine and could live 6.9 months in average. The others who did not respond to the medicine only survive for 1.8 months.

Modem medical science is constantly searching for safer and more effective medical herbs to treat cancers. Plants of the family Euphorbiaceae, particularly the genus Euphorbia, have been used to treat cancers, tumors and warts for hundreds of years (Lloydia, 32, 157-176, 1969). Euphorbia esula L. and Croton tiglium L., two members of family Euphorbiacease, have been used widely in folk medicines for treating cancers. Ingenol 3,20-debenzoate, the extract isolated from Euphorbia esula L., and phorbol 12-tiglate 13-decanoate, the extract isolated from Croton tiglium L., showed antileukemic activity against the P-388 lymphocytic leukemia in mice. The extract from seeds of Euphorbia lagascae Spreng also shows inhibitory activity against several 9PS and 3PS (P-388) leukemia in marine (J. Nat. Prod., 47:347, 1984). Euphorbia Kansui has been used as herbal remedy for edema, ascites and cancer in China. Two antileukemic diterpene esters extracted from the roots of Euphorbia Kansui.

The 13-hydroxyingenol-3-(2,3-dimethylbutanoate)-13-dodecanoate-20-hexadecanoate, and 6,7-epoxy-13-hydroxyingenol-3-(2,3-dimethylbutanoate)-13 dodecanoate-20-hexadecanoate have been isolated and confirmed with antileukemic effect against P-388 lymphocytic leukemia in the mice (J. Nat, Prod., 54(3), 823-829, 1991). Two cytotoxic triterpenes, 9,19-cycloart-23-ene-3 β,24-diol, have been isolated from the leaves of Euphorbia pulcherrima Willd and demonstrated significant inhibitory activity against Ehrlich acites tumor cell (Planta Med. 62, 322-5, 1996). A number of tigliane diterpene esters from the latex of Euphorbia poisonii, a highly-toxic plant found in Northern Nigeria can be used as garden pesticide and is reputed to be used in homicides. One of these compounds shows a selective cytotoxicity for the human kidney carcinoma cell line A-498 more than 10,000 times greater than that of adriamycin (J. Med. Chem., 1996, 39(4): 1005-1008).

Thus, while there are numerous reports of anti-cancer activity of various Euphorbia preparations, there is still a need to find out a species of Euphorbia that not only has an anti-cancer effect but also has low toxicity.

### SUMMARY OF THE INVENTION

It is surprisingly found in the invention that the extract of plants from Euphorbia antiquorum specifically inhibits growth of five different human cell lines, including human hepatoma cell line, human colorectal adenocarcinoma cell line, human monocyte-like lymphoma and human leukemia T cell. Moreover, the extract from Euphorbia antiquorum induces morphological changes of colorectal adenocarcinoma cells. Furthermore, when compared to other species which have been recognized by anti-cancer activity, the extract from Euphorbia antiquorum has very low toxicity.

In a first aspect, the invention provides an extract from the species Euphorbia antiquorum.

In a second aspect, the invention provides a pharmaceutical composition comprising the extracts together with a pharmaceutically acceptable carrier or diluent.

In a third aspect, the invention provides a method of treatment of a cancer in a subject, comprising the step of administering a therapeutically effective amount of an extract of the invention to said subject. Preferably, the cancer is hepatoma, leukemia, colon cancer or lymphocytoma.

In a fourth aspect, the invention provides a method of inhibiting proliferative activity of tumor cells, comprising the step of exposing the cells to an anti-proliferative amount of an extract of the invention. The cells may be treated either in vivo or in vitro.

### BRIEF DESCRIPTION OF THE DRAWINGS

FIG. 1 shows an acute oral toxicity study of Gerent-Tu-A in ICR mice—mean body weight growth curved.

FIG. 2 demonstrates a concentration-cytotoxicity curve of MH801 against human colon tumor cells COLO 205.

FIG. 3 demonstrates a concentration-cytotoxicity curve of MH801 against human colon tumor cells HT-29.

FIG. 4 demonstrates a concentration-cytotoxicity curve of MH801 against human colon tumor cells U937.

FIG. 5 demonstrates a concentration-cytotoxicity curve of MH801 against human colon tumor cells MOLT 4.

FIG. 6 demonstrates a concentration-cytotoxicity curve of MH801 against human colon tumor cells HA22T/VGH.

### DETAILED DESCRIPTION OF THE INVENTION

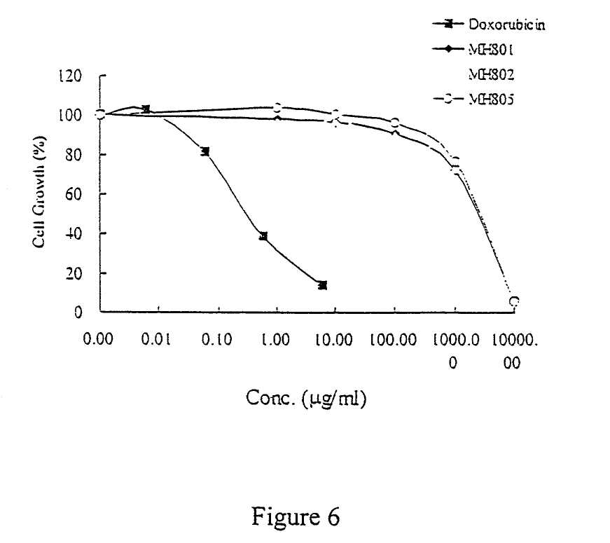

FIG. 6 demonstrates a concentration-cytotoxicity curve of MH801 against human colon tumor cells HA22T/VGH. The present invention relates to a novel discovery that an herbal extract of Euphorbia antiquorum can effectively inhibit tumor growth and is substantially nontoxic when administered to an individual. “Inhibiting tumor growth” is used herein, for purposes of the specification and claims, to mean slowing down the growth of the tumor, halting growth of the tumor, causing reduction or regression of the tumor, causing morphological changes of tumor cells.

Euphorbia antiquorum is a member of the family Euphorbiaceae. Euphorbia antiquorum is an armed plant with stiff branches bearing stiff spines. The height of plant is from about one to several meters. The flowers have a slight unpleasant smell that attracts many varieties of insects. The cortex is gray and has a slight rift. When the outer skin of the plant is scratched, there is milky exudates effusion. The odor of this latex is pungent and lingering. The latex is irritating to the skin, and the sap is irritating to the human eye. It even blinds one if contacting the eye directly.

In China, Euphorbia antiquorum is also termed huoyangle, bawanbian, jingangtsuan, jinganshu, huoshiang, chuenyangtsau, alishu, yangbukai, lunggutsz, huoyan, laigetsz, chousung, taigetsz, huohung, chiannianjian, rouchilin, yanbuai, huowang, yana, shiauchinglung, chaibutung, baibuhueiyan, yuanjingan or meitzadajan. This plant is distributed in Zhejiang, Jujian, Guangdong, Guangxi, Sichuan, Hainan, Guizhou, Yunan and Taiwan (Republic of China). In Taiwan, these Euphorbia antiquorum usually bloom from April to May. The height of this plant is about two to three meters, or up to six to seven meters.

All parts of this plant have been frequently used in indigenous systems of medicine for a long time. It can relieve cold, detoxify and reduce inflammation. The major function is treatment for skin tinea and edema. Previous investigations on this plant showed the presence of taraxerol and epi-friedelanol in the stem-bark; friedelan-3 β-ol and 3 α-ol, taraxerol and taraxerone in the sterms; euphol, euphorbol, β-amyrin, cycloartenol and ingenol type diterpenoids in the latex (Indian Medicinal Plants Part III, 2202, 1993).

The invention provides an extract from the species Euphorbia antiquorum. Typically, the fresh or dried leaves, seeds, bark, fruit, peel and flowers of the plant are suitable for in preparation of the herb extract. Generally the herb is extracted with hot water.

Experimental results (discussed below) indicate that the herbal extract of the invention is effective in inhibiting the growth of tumor cells. Among the advantages of the invention is that the herb extracts are able to induce differentiation in colorectal adenocarcinoma cells so that morphological appearance of such cells have been changed. Furthermore, according to the Acute Oral Toxicity Experiment, the volume of the acute oral toxicity test LD₅₀ is higher than 5,000 mg/Kg, which is relatively higher than that of other species of family Euphorbiaceae. These experiments and the resulting data demonstrate the herbal extract from Euphorbia antiquorum according to the present invention is a useful source to treat cancers and satisfies the need for a herb extract with low toxicity.

The invention also provides a pharmaceutical composition comprising the extracts together with a pharmaceutically acceptable carrier or diluent. The carrier or diluent will depend on the route of administration, and persons skilled in the art will readily be able to determine the most suitable formulation for each particular case. It is contemplated that compositions of the invention may be administered orally and/or by parenteral injection, including intravenous injection.

Methods and pharmaceutically acceptable carriers for preparation of pharmaceutical compositions are well known in the art.

The invention further provides a method of treatment of a cancer in a subject, comprising the step of administering a therapeutically effective amount of an extract of the invention to said subject. Preferably, the cancer is hepatoma, leukemia, colon cancer or lymphocytoma. The compositions of the invention can be administered through any suitable route. Persons skilled in the art will readily be able to determine the most suitable route and dose for the conditions to be treated. Dosages will be at the discretion of the attendant physician or veterinarian, and will depend on the nature and state of the condition to be treated, the age and general state of health of the subject to be treated, the route of administration, and any previous treatments that may have been administered.

The subject may be a human, or may be a domestic or companion animal. While it is particularly contemplated that the extracts or compositions of the invention are suitable for use in medical treatment of humans, it is also applicable to veterinary treatment.

Furthermore, the invention provides a method of inhibiting proliferative activity of tumor cells, comprising the step of exposing the cells to an anti-proliferative amount of an extract of the invention. The cells may be treated either in vivo or in vitro.

The invention will be described in detail by reference to the following non-limiting examples. As used herein, the following nomenclature, MH801 will be used to identify a single Chinese herbal medicine of Euphorbia antiquorum. The herb used as starting material for the invention may be obtained from commercial sources. The plant extracts, once isolated from the plant material, may be concentrated and then placed in form suitable for the administration to a subject (i.e., pills, capsules and tablets).

### EXAMPLE 1

### Preparation of Herb Extract from Euphorbia antiquorum

The process described below can be scaled up to produce larger quantities of extracts. The details of the process for preparation of Euphorbia antiquorum extract described below should not be considered as limiting. The quantities and times described below can be varied substantially to provide suitable extract of Euphorbia antiquorum in accordance with the invention.

The suitable part or parts or the herb of a medicinal plant Euphorbia antiquorum was obtained, washed with cold water, dried and comminuted. The plant materials were then extracted with boiling water on a basis of 1 part by weight of plant material to approximately 5 to 10 parts by weight of water. The amount of water used should at least cover the plant material in the extraction vessel. The mix therein was decocted at 100 C. for about thirty minutes, but not in excess of one hour so as to allow effective extraction of the desired components. The mixture was allowed to cool to room temperature to provide a liquid extract of Euphorbia antiquorum. The aqueous solution of Euphorbia antiquorum was separated from the plant material by filtration.

The decoction may be directly consumed after it is prepared and cooled to warm or ambient temperature or preserved with proper sterilization for later consumption. Sterilization may be accomplished by microfiltration or heating. The aqueous solution may be freezing dried or spraying dried, or reduced in volume by heating with or without an applied vacuum. The concentrate may then by spraying dried or freezing dried to absorbed by powdered material of the same plant material or starch and thus the single-herb extract is prepared in powdered form.

### EXAMPLE 2

### Acute Oral Toxicity Study

This example describes the acute toxicity of MH801 according to the invention prepared from Example 1 at a dose level of 5,000 mg/kg (hereafter called Gerent-Tu-A). The test article of the invention, Gerent-Tu-A, was suspended in injection grade water to make the concentrate of 500 mg/ml. Twenty-four ICR mice were divided into two groups (six males and six females per group) in this study. Twelve tested mice were each fed with Gerent-Tu-A(500 mg/ml) of the invention, while the other mice were fed with vehicle (injection grade water only) as control, by gavage, at a volume of 10 ml/kg. During the 14-day study observation period, there was no mortality and clinical signs observed in the treated and control mice. There was no difference in body weight gains between the treated and control groups. Necropsy of all the mice at the termination of the 14-day study did not reveal any gross lesions. The experimental results indicated that ingestion of Gerent-Tu-A, at a dose level of 5,000 mg/kg according to the invention, did not cause any observable acute pharmacotoxic effects in treated ICR mice (Table 1). The no observable effect level (NOEL) was therefore estimated to be 5,000 mg/kg. Based on this single dose NOEL, the test article Gerent-Tu-A of the invention may be classified as “practically nontoxic” according to Loomis (Loomis TA. Essentials of Toxicology. Philadelphia: Lea & Febiger, 1978).

TABLE 1
Acute Oral Toxicity Study of Gerent-Tu-A in ICR Mice

| | Dose | Mortality | Clinical | Gross Necropsy |
|---|---|---|---|---|
| Sex | (mg/kg) | N/N | Signs | Findings |
| Male | 0 (vehicle control) | 0/6 | NO | NOL |
| | 5,000 | 0/6 | NO | NOL |
| Female | 0 (vehicle control) | 0/6 | NO | NOL |
| | 5,000 | 0/6 | NO | NOL |

N/N = Number of mice found dead/Number of mice examined
NO = Not observed
NOL = No observable lesions found

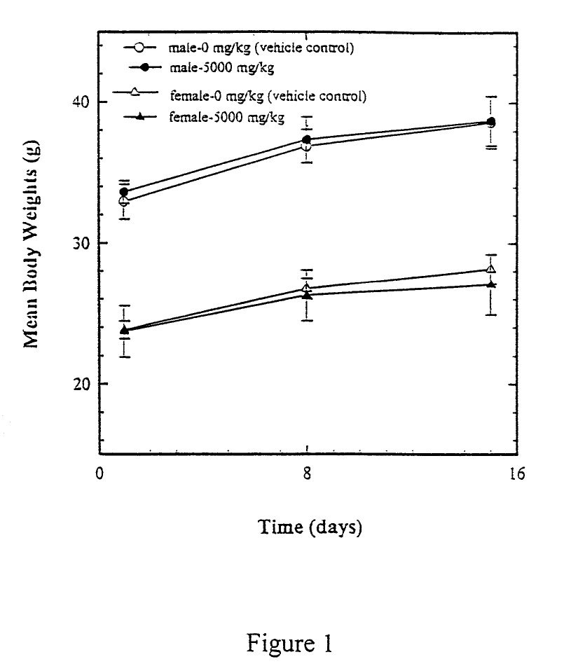

The mean body weight of each group is presented in Table 2, and the mean body weight growth curves are also shown in FIG. 1. There are no differences in mean body weights as well as body weight gains between treated and control mice.

TABLE 2
Acute Oral Toxicity Study of Gerent-Tu-A in ICR Mice - Mean Body
Weights and Weight Gains (Mean ± SD, N = 6)

| | Dose | Mean Body Weights (g) | | Weight Gains (g) | |
|---|---|---|---|---|---|
| Sex | (mg/kg) | Initial (SD1) | SD 8 | Final (SD15) | On SD15 |
| Male | 0 | 32.93 ± 1.23 | 36.88 ± 1.20 | 38.62 ± 1.82 | 5.68 ± |
| | (Vehicle) | | | | 1.32 |
| | 5,000 | 33.62 ± 0.81 | 37.35 ± 1.62 | 38.75 ± 1.76 | 5.13 ± |
| | | | | | 1.75 |
| Fe- | 0 | 23.83 ± 0.62 | 26.75 ± 0.74 | 28.17 ± 1.07 | 4.33 ± |
| male | (Vehicle) | | | | 0.87 |
| | 5,000 | 23.72 ± 1.82 | 26.28 ± 1.81 | 27.07 ± 2.14 | 3.35 ± |
| | | | | | 0.67 |

There were no treatment-related differences observed in mortality, clinical signs, body weight gains and gross changes in this study, when Gerent-Tu-A of the invention was administered orally to ICR mice at a single dose of 5,000 mg/kg, followed by 14 days of observation.

### EXAMPLE 3

### Inhibitory Activity of Euphorbia antiguorum Extract Against Tumor Cell Lines

The ability of extract of Euphorbia antiquorum described in Example 1 to inhibit the growth of five different human tumor cell lines was tested. The activity of Doxorubicin, a currently clinically used chemotherapeutic agent, against the tumor cell lines was tested as a control.

### Tumor Cell Lines

Five human tumor cell lines COLO 205, HT-29, U-937, MOLT4 and HA22T/VGH, were tested in this example (Table 3).

TABLE 3
Cell Line Description

| Cell Line | Description | Source |
|---|---|---|
| COLO 205 | Human colorectal adenocarcinoma cell line | CCRC^{a} 60054 |
| HT-29 | Human colorectal adenocarcinoma cell line | ATCC^{b}HTB-38, HTB-52 |
| U-937 | Human monocyte-like lymphoma | CCRC 68002 |
| MOLT 4 | Human leukemia; T cell | CCRC 60060 |
| HA22T/VGH | Human hepatoma cell line | CCRC 60168 |

CCRC^{a} means American Type Culture Collection, Rockville, MD, USA.
ATCC^{b} means Culture Collection and Research Center, Hsinchu, Taiwan.

The result of mycoplasma test of the five cancer cell lines is negative. All five cancer cell lines were stored in liquid nitrogen. After thawing, cells were maintained as monolayer in appropriate medium (GIBCO, USA) supplemented with 10% fetal bovine serum, sodium bicarbonate, L-glutamine (2 mM), streptomycin (100 μg/ml) and penicillin-G (100 units/ml) in a humidified incubator at 37±1° C. and 5% CO₂ in air.

### Preparation of Target Cells

Exponentially growing human cancer cells were seeded in an appropriate medium in 96-well ELISA microtiter plates. The number of cells for each cell line plated in 96-well plates was determined from the growth curve obtained in MTS assay (600 to 20,000 cells depending on cell line). These plates of adherent cells were incubated overnight. Six well cultures were used for each concentration of MH801 of the invention and for negative and positive control cultures.

### Test Substance Preparation

MH801 was dissolved in culture medium. In this experiment, MH801 was not completely soluble in culture medium or DMSO in the highest test concentration. Since the solution prepared in culture medium at working concentration can be used directly rather than a stock solution at 200 fold concentration in DMSO, culture medium, instead of DMSO, was used as vehicle in the present invention. Maximum concentration for this assay was 10 mg/ml. Serial ten fold dilution was used in this invention, and therefore, the concentrations were 10, 1, 0.1, 0.01 and 0.001 mg/ml. After sufficient mixing, this solution was passed through filter of 0.2 μm pore size for sterility. Filtrate was serially diluted with culture medium to desired concentrations for treatment. Solution of test article at the highest concentration maintains pH at 7.0.

### Cell Proliferation Assay

After three days of culture, the viable cells were counted by changing the culture medium containing a tetrazolium compound

(3-4,5-dimethylthiazol-2-yl)-5(3-carboxymethoxyphenyl)-2-(4-sulfophenyl)-2H-tetrazolium, inner salt and an electron coupling reagent to measure mitochondrial enzyme activity. The absorbance at 490 μm was measured after 2 hours to 4 hours reaction time. Cell growth % is calculated using the following equations:

$$Cell growth \% = mean of {A}_{490 nm} of treated group \text{/} mean of {A}_{490 nm} of negative control group \times 100.$$

Doxourbicin was used as positive control for assay system evaluation and was prepared in dimethyl sulfoxide (Merck Art. 2931, Germany) as stock solution to be added to culture medium.

### Results

The inhibitory effect of MH801 of the invention on the proliferation of 5 human tumor cells is shown in Table 4.

TABLE 4
Effect of MH801 on Growth of Five Human Tumor Cells

| | Treatment | Cell Growth | | | | |
|---|---|---|---|---|---|---|
| | (μg/ml) | COLO 205 | HT-29 | U-937 | MOLT 4 | HA22T/VGH |
| MLH801 | untreated | 100 | 100 | 100 | 100 | 100 |
| | 1 | 102 | 104 | 102 | 103 | 98.6 |
| | 10 | 104 | 103 | 102 | 105 | 96.8 |
| | 100 | 106 | 106 | 103 | 76.7 | 90.9 |
| | 1000 | 59.9 | 99.1 | 113 | 43.6 | 72.7 |
| | 10000 | 14.8 | 7.97 | 16.5 | 19.8 | 7.03 |
| Doxorubicin | 0.0058 | — | — | 100.5 | 71.2 | 103 |
| | 0.058 | 63.2 | — | 80.9 | 0 | 81.53 |
| | 0.58 | 33.8 | — | 3.7 | 0 | 38.8 |
| | 1.74 | — | 35 | | | — |
| | 3.48 | — | 15.9 | | | — |
| | 5.8 | 8.23 | — | 1.6 | 0 | 14.4 |

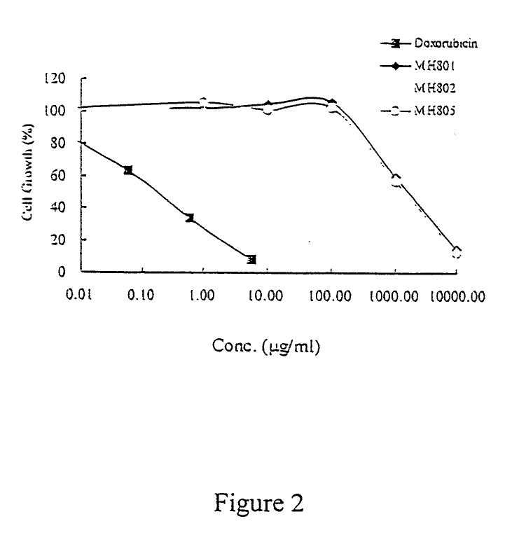

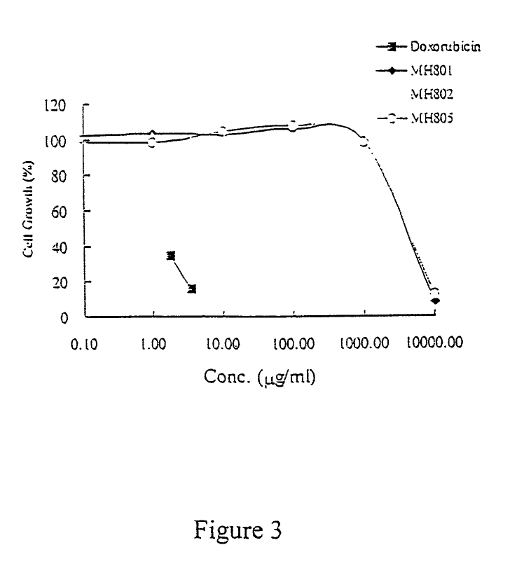

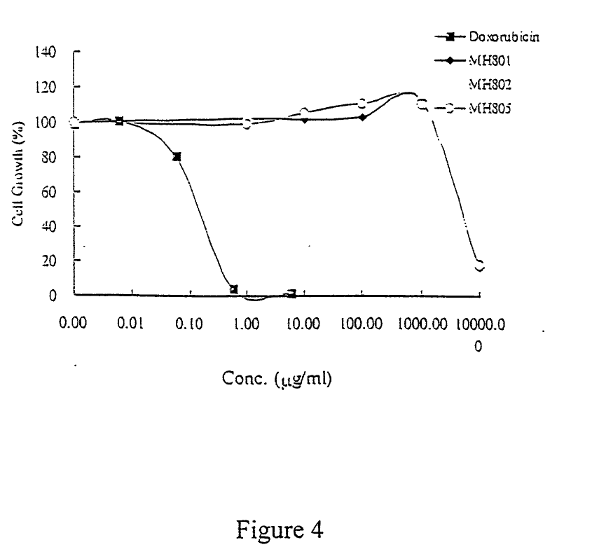

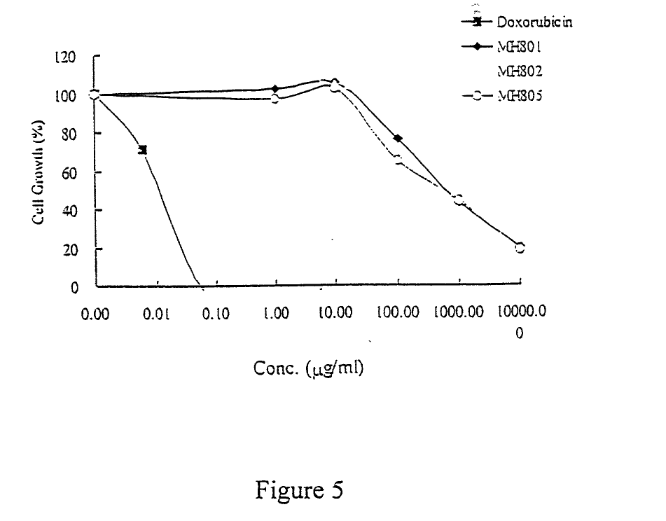

To give a clear comparison of the activity, the concentration-inhibition curve for each tumors are also shown in the FIGS. 2 to 6. In all five tumors, the highest concentration of MH801 exhibits strong inhibition about 75 to 95% mark. A clear dose-response effect is demonstrated especially in 2 tumors, COLO 205 and MOLT 4. The concentration required to produce 50% inhibition (IC₅₀) are about 1 mg/ml for both tumors. There is a steep slope in dose-response effect in the other three tumors, with second highest concentration (1 mg/ml) showing almost no antiproliferative effect but strong cytotoxicity induced at 10 mg/ml. The factors contributing to this heterogeneity between tumors involve different level of sensitivity of each tumor and different metabolic rate within each tumor.

Doxorubicin, a currently clinically used chemotherapeutic agent as a positive control, exhibits very strong antiproliferative effect against all five tumors in this study.

During the exposure of the tumors to MH801 of the invention, a distinct morphology change of the treated tumor cells was noticed. When tumor cells treated with MH801 at concentration of 0.1 mg/ml, a few fibroblast-like cells gradually increased but the untreated tumors remain rounded in shape. At concentration of 1 mg/ml, more fibroblast-like cells were induced, and more than 40% of cell population changing into fibroblasts were estimated for COLO 205 cell line. This phenomenon is particularly obvious for COLO 205, MOLT 4 and U-937 cells because these three cell lines were round in shape normally when observed under microscope. For HT-29 and HA22T/VGH, less aggregation of the cells was observed when treated with MH801. Many fibroblast-like cells appeared on exposure to 1 mg/ml of MH801 while the negative control kept COLO 205 in its original round shape. Cell death was observed at 10 mg/ml of treatment group. After 70 hours of incubation there was a marked reduction in the number of COLO cells and a pronounced change in their morphology. The cells had reverted to a long, spindly appearance. Since the COLO 205 cells appeared to have adopted this altered morphology, which is unexpected given the heterogeneous nature of the fibroblasts. A further study of the mechanism behind this morphology change and its implication in anti-tumor activity is needed.

In conclusion, MH801 according to the invention exhibits anti-proliferative effect against 5 human tumor cells including 2 colon tumors, COLO 205 and HT-29, 2 leukemia/lymphoma, U-937 and MOLT 4, and one hepatoma, HA22T/VGH. In other words, the extracts from Euphorbia antiquorum of the invention are useful in effective treatment of cancers, particularly hepatoma, leukemia, colon cancer or lymphocytoma.

In an alternative, the method according to the present invention for anticancer therapy further comprises administering a therapeutically effective amount of one or more standard anticancer treatments (e.g., one or more of radiation therapy, chemotherapy, surgery, immunotherapy, photodynamic therapy, and a combination thereof) in addition to administering a therapeutically effect amount of the extracts or compositions containing the same of the invention.

As known to those skilled in the art, the dosage may vary with the individual depending on the age, size and health condition of the individual, and related factors. The route of administration may be by any conventional route in which the extracts or compositions can be safely and effectively delivered. A preferred route of administration is an oral route. The composition may be administered in tablet, capsule form, or in a form in a pharmaceutically acceptable carrier (e.g., liquid, water, saline or other physiological solution, or gel).

From the above, it will be obvious to those skilled in the art that the examples described above are offered by way of illustration of the use of the extract from Euphorbia antiquorum for treatment of cancers in this subject application and not by way of limitation. Accordingly, the above-described examples are for the purpose of clarity and understanding of the present invention; various modifications and alterations to the embodiments and methods may be made without departing from the scope of the inventive concept disclosed in this specification.

What we claim are:

1. An herbal extract from Euphorbia antiquorum for inhibiting tumor growth.

2. A pharmaceutical composition for treatment of cancers comprising an herbal extract from Euphorbia antiquorum and pharmaceutically acceptable carrier or diluent.

3. A method of treatment of a cancer in a subject comprising administering to said subject a therapeutically effective amount of an extract from Euphorbia antiquorum or a pharmaceutical composition comprising the extract from Euphorbia antiquorum.

4. The method of claim 3, wherein said subject is a human.

5. The method of claim 3, wherein the cancer is selected from the group consisting of hepatoma, colon cancer, leukemia and lymphocytoma.

6. The method of claim 3, further comprising administering to the subject a therapeutically effective amount of one or more anticancer treatment selected form the group consisting of radiation therapy, chemotherapy, surgery, immunotherapy, photodynamic therapy, and a combination thereof.

7. The method of claim 3, wherein the extract or pharmaceutical composition is administered orally to the subject.

8. A method of inhibiting tumor growth comprising contacting cells with an effective amount of an herbal extract from Euphorbia antiquorum.

9. The method of claim 8 which is applied in vitro or in vivo.

10. The method of claim 8, wherein said cells comprise cells from normal patients.

11. The method of claim 10, wherein said cells comprise cells from patients having hepatoma.

12. The method of claim 10, wherein said cells comprise cells from patients having colon cancer.

13. The method of claim 10, wherein said cells comprise cells from patients having leukemia.

14. The method of claim 10, wherein said cells comprise cells from patients having lymphocytoma.
